(19) United States
(12) Patent Application Publication
(10) Pub. No.: US 2026/0076647 A1
(43) Pub. Date: Mar. 19, 2026

(54) ECHOCARDIOGRAM UNIT FOR MECHANICAL ASSIST DEVICES

(71) Applicant: Ultrasight Ltd., Nes Ziona (IL)

(72) Inventors: Itay KEZURER, Rehovot (IL); Jonathan SHLOMI, Givat Ada (IL)

(21) Appl. No.: 19/328,242

(22) Filed: Sep. 15, 2025

Related U.S. Application Data
(60) Provisional application No. 63/695,375, filed on Sep. 17, 2024.

(51) Int. Cl. A61B 8/00; A61B 8/04; A61B 8/08; A61B 90/00

(52) U.S. Cl. CPC A61B 8/4245; A61B 8/04; A61B 8/0883; A61B 8/463; A61B 8/5207; A61B 8/58; A61B 2090/3925

(57) ABSTRACT

An ultrasound scanning unit for assessing an implanted device within a body part includes a structural reference identifier and a structural reference measurer. The structural reference identifier receives one or more ultrasound images of a heart. From the images, it identifies and locates an anatomical structural reference, such as an aortic annulus, and a device structural reference, such as an inlet of a mechanical assist device. The structural reference measurer calculates a spatial relationship, like a distance, between the identified references to provide a positional assessment of the implanted device. The unit can also include a navigator to generate orientation instructions for guiding a user in positioning an ultrasound probe. A mal-rotation detector determines if the device has an incorrect rotational orientation, and a safe zone generator creates a map of safe and unsafe positions. A pressure-volume loop determiner also generates a pressure-volume loop to assess myocardial contractility.

### CROSS REFERENCE TO RELATED APPLICATIONS

This application claims priority from U.S. provisional patent application 63/695,375, filed Sep. 17, 2024, which is incorporated herein by reference.

### FIELD OF THE INVENTION

The present invention relates to echocardiogram systems generally and to echocardiogram systems for mechanical assist devices in particular.

### BACKGROUND OF THE INVENTION

Mechanical circulatory support devices, such as intravascular blood pumps, are critical for providing temporary hemodynamic support to patients. These devices are often used in high-risk clinical scenarios, such as for patients experiencing cardiogenic shock or undergoing high-risk percutaneous coronary interventions (PCI). One such example is the Impella® device, by Abiomed, which is designed to reduce the workload of the heart and improve systemic circulation by actively pumping blood from the left ventricle into the ascending aorta.

Reference is now made to FIGS. 1A and 1B, which is a schematic illustration of a mechanical assist device 10 and its intended placement within a heart 20. FIG. 1A shows an exemplary mechanical assist device 10, illustrating key components such as an inlet 12, a radiopaque section 14, and a microaxial motor 16. FIG. 1B illustrates the intended placement of the device 10, which is positioned to extend from the left ventricle 22, across the aortic valve 26, and into the ascending aorta 24. In this configuration, the device 10 pumps blood from the inlet 12 in the left ventricle 22 to an outlet in the aorta 24.

The correct positioning of such mechanical assist devices is paramount for optimizing blood flow and avoiding complications. Improper positioning may result in a loss of mechanical support or other adverse events. Consequently, the initial placement of the device is typically confirmed using imaging, such as fluoroscopy, in a catheterization laboratory. Subsequent verification of the device's position, especially after the patient has been moved or in response to device alarms, is often performed at the patient's bedside using transthoracic echocardiography (TTE).

Reference is now made to FIGS. 2A and 2B, which illustrate a Parasternal Long-Axis (PLAX) echocardiographic view, both without and with the mechanical assist device 10, respectively. FIG. 2A shows a standard PLAX view of a heart, indicating the location of the left ventricle 22 and the aorta 24. FIG. 2B shows a similar view but with the mechanical assist device 10 present. As indicated, parts of the mechanical assist device are visible, including a portion 10A in the left ventricle and a portion 10B in the aorta. This view is critical for confirming that the device components are situated correctly relative to the heart's anatomy.

### SUMMARY OF THE PRESENT INVENTION

There is therefore provided, in accordance with a preferred embodiment of the present invention, a unit for assessing a position of an implanted device within a body part, the unit including a structural reference identifier. The structural reference identifier receives one or more ultrasound images, and identifies and locates therefrom, at least one anatomical structural reference of the body part and at least one device structural reference of the implanted device.

Moreover, in accordance with a preferred embodiment of the present invention, the unit also includes a structural reference measurer which calculates a spatial relationship between the at least one anatomical structural reference and the at least one device structural reference to provide a positional assessment of the implanted device.

Further, in accordance with a preferred embodiment of the present invention, the implanted device is a mechanical assist device, the body part is a heart, the at least one anatomical structural reference is an aortic annulus, the device at least one structural reference is an inlet of the mechanical assist device, and the spatial relationship is a distance.

Still further, in accordance with a preferred embodiment of the present invention, the structural reference identifier includes a machine learning model trained to identify and locate the at least one anatomical and device structural references and to determine a visibility level for the anatomical and device structural references.

Additionally, in accordance with a preferred embodiment of the present invention, the unit further includes a validator which receives the one or more ultrasound images as a sequence of frames and permits the structural reference measurer to measure the references only when the at least one anatomical structural reference and the at least one device structural reference are determined to both be visible for a predefined duration within the sequence of frames.

Moreover, in accordance with a preferred embodiment of the present invention, the unit further includes a display module. The display module generates a graphical overlay on the one or more ultrasound images, the overlay including an indication of the spatial relationship.

Further, in accordance with a preferred embodiment of the present invention, the unit further includes a display module. The display module generates a graphical overlay on the one or more ultrasound images, the overlay including a first marker indicating a location of the identified anatomical structural reference and a second marker indicating a location of the identified device structural reference.

Still further, in accordance with a preferred embodiment of the present invention, the unit further includes an ultrasound navigator. The ultrasound navigator analyzes a stream of the one or more ultrasound images and generates orientation instructions for guiding a user to position an ultrasound probe to acquire a view close to a canonical view of the body part.

Additionally, in accordance with a preferred embodiment of the present invention, the unit further includes a mal-rotation detector. The mal-rotation detector identifies one or more surrounding anatomical structures of a left ventricle of the heart within the one or more ultrasound images and determines whether or not a rotational and/or positional orientation of the implanted device is properly rotated and oriented within the left ventricle.

Moreover, in accordance with a preferred embodiment of the present invention, the unit further includes a safe zone generator trained to identify and delineate boundaries of the left ventricle wall. The safe zone generator generates a map of zones overlaid on the one or more ultrasound images, the map of zones indicating safe and unsafe positions for the implanted device.

Further, in accordance with a preferred embodiment of the present invention, the unit further includes a left ventricle volume determiner and a pressure-volume loop determiner. The left ventricle volume determiner generates a time-stamped volume of a left ventricle, marked with cardiac cycle events, from the one or more ultrasound images. The pressure-volume loop determiner synchronizes the time-stamped volume with received time-stamped pressure data from the mechanical assist device, generates a pressure-volume loop therefrom, and marks the loop with the cardiac cycle events.

Still further, in accordance with a preferred embodiment of the present invention, the unit is implemented on a mobile computing device having a display to display the one or more ultrasound images and is mountable at a point of care near a patient having the device implanted therein.

Additionally, in accordance with a preferred embodiment of the present invention, the mobile computing device includes a barcode scanning functionality to associate data in a database with the patient having the device.

Moreover, in accordance with a preferred embodiment of the present invention, the structural reference identifier operates on the one or more ultrasound images in real-time or offline.

There is therefore provided, in accordance with a preferred embodiment of the present invention, a modular mounting assembly for a mobile display device, the assembly including a mounting plate, a case, and a clamp component. The case is detachably coupled to the mounting plate and houses the mobile display device. The clamp component is attached to the mounting plate and engages a medical pole.

Further, in accordance with a preferred embodiment of the present invention, the assembly also includes a transducer holder integrated with the mounting plate, and the transducer holder receives an ultrasound probe.

There is therefore provided, in accordance with a preferred embodiment of the present invention, a method for assessing a position of an implanted device within a body part. The method includes receiving one or more ultrasound images of the body part with the implanted device, and identifying and locating, from the one or more ultrasound images, at least one anatomical structural reference of the body part and at least one device structural reference of the implanted device.

Still further, in accordance with a preferred embodiment of the present invention, the method further includes calculating a spatial relationship between the at least one anatomical structural reference and the at least one device structural reference to provide a positional assessment of the implanted device.

Additionally, in accordance with a preferred embodiment of the present invention, the calculating of the spatial relationship includes calculating a distance between an aortic annulus of a heart and an inlet of a mechanical assist device implanted therein.

Moreover, in accordance with a preferred embodiment of the present invention, the identifying and locating are performed using a trained machine learning model, and the method further includes determining, using the trained machine learning model, a visibility level for the anatomical and device structural references.

Further, in accordance with a preferred embodiment of the present invention, the method further includes receiving the one or more ultrasound images as a sequence of frames, and permitting the calculating of the spatial relationship only when the determined visibility level indicates that the at least one anatomical structural reference and the at least one device structural reference are both visible for a predefined duration within the sequence of frames.

Still further, in accordance with a preferred embodiment of the present invention, the method further includes generating a graphical overlay on the one or more ultrasound images, the overlay including an indication of the calculated spatial relationship.

Additionally, in accordance with a preferred embodiment of the present invention, the method further includes generating a graphical overlay on the one or more ultrasound images, the overlay including a first marker indicating a location of the identified anatomical structural reference and a second marker indicating a location of the identified device structural reference.

Moreover, in accordance with a preferred embodiment of the present invention, the method further includes analyzing a stream of the one or more ultrasound images, and generating orientation instructions for guiding a user to position an ultrasound probe to acquire a view close to a canonical view of the body part.

Further, in accordance with a preferred embodiment of the present invention, the method further includes identifying one or more surrounding anatomical structures of a left ventricle of the heart within the one or more ultrasound images, and determining whether a rotational or positional orientation of the mechanical assist device is properly rotated and oriented within the left ventricle.

Still further, in accordance with a preferred embodiment of the present invention, the method further includes identifying and delineating boundaries of a left ventricle wall from the one or more ultrasound images, and generating a map of zones overlaid on the one or more ultrasound images, the map of zones indicating safe and unsafe positions for the mechanical assist device.

Additionally, in accordance with a preferred embodiment of the present invention, the method further includes generating a time-stamped volume of a left ventricle, marked with cardiac cycle events, from the one or more ultrasound images, receiving time-stamped pressure data from the mechanical assist device, synchronizing the time-stamped volume with the received time-stamped pressure data, generating a pressure-volume loop from the synchronized data, and marking the loop with the cardiac cycle events.

Moreover, in accordance with a preferred embodiment of the present invention, the method further includes displaying the one or more ultrasound images on a mobile computing device at a point of care near a patient having the device implanted therein.

Further, in accordance with a preferred embodiment of the present invention, the method further includes scanning a barcode to associate data in a database with the patient.

Finally, in accordance with a preferred embodiment of the present invention, the identifying and locating are performed on the one or more ultrasound images in real-time or offline.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B are prior art schematic illustrations of a mechanical assist device and its intended placement within a heart;

FIGS. 2A and 2B are prior art screen shot illustrations of a Parasternal Long-Axis (PLAX) echocardiographic view without and with a mechanical assist device, respectively;

FIG. 3 is a combined block diagram and schematic illustration of a system for providing ultrasound for mechanical assist devices, constructed and operative according to an embodiment of the present invention;

FIG. 4 is a schematic illustration showing the data flow within the system of FIG. 3;

FIGS. 5A, 5B, and 5C are schematic illustrations depicting an exemplary operational workflow of the system of FIG. 3;

FIG. 6 is a block diagram illustration showing a detailed architecture of a structural reference identifier forming part of the system of FIG. 3;

FIG. 7 is a block diagram illustration showing a detailed architecture of a measurer forming part of the system of FIG. 3;

FIG. 8 is a combined block diagram and schematic illustration of an alternative system for providing ultrasound for mechanical assist devices;

FIGS. 9A and 9B are, respectively, front and back expanded view illustrations of a modular mounting assembly useful in the system of FIG. 8;

FIG. 10 is a schematic illustration of a device identification function useful in the systems of FIGS. 3 and 8;

FIG. 11 is a combined block diagram and schematic illustration of another alternative system for providing advanced positional insights;

FIG. 12 is a screen shot illustration showing exemplary images of optimal and suboptimal device positioning identifiable by the system of FIG. 11;

FIG. 13 is a screen shot illustration showing an exemplary safe zone output generated by the system of FIG. 11;

FIG. 14 is a combined block diagram and schematic illustration of a system configured for automated contractility assessment; and

FIG. 15 is a combined schematic and graphical illustration showing the input to and output from a pressure-volume loop determiner useful in the system of FIG. 14.

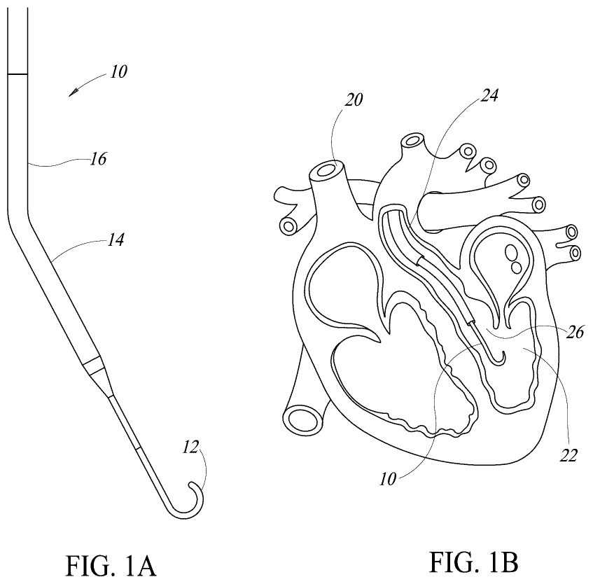

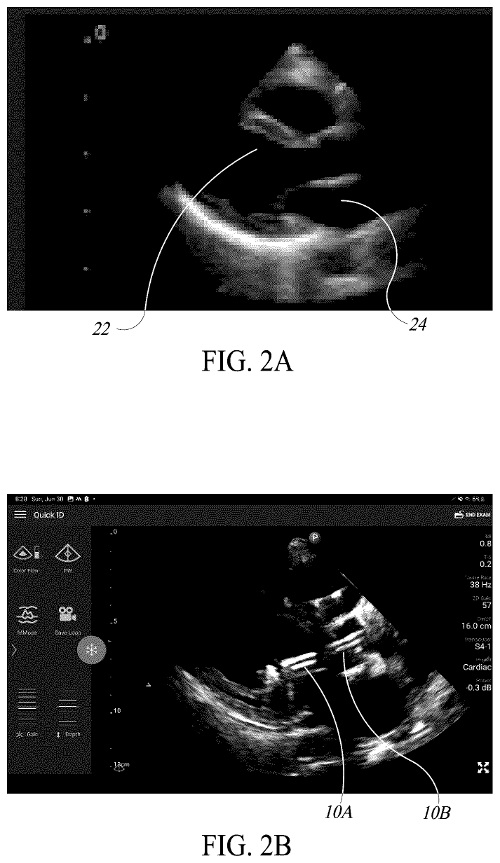

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that a significant gap exists in the standard of care for patients with mechanical assist devices. While echocardiography is essential for both routine monitoring and emergency assessment of the device's position, its use is critically limited by the availability of personnel with specialized training in sonography. In many clinical settings, such as an Intensive Care Unit (ICU), a trained sonographer or echocardiographer is not available 24/7. This scarcity creates a challenge for the medical team responsible for managing these patients, potentially delaying the assessment required to respond to device alarms or to verify correct positioning, thereby increasing the risk of complications associated with device malposition.

Applicant has further realized that this critical gap can be addressed by a system that empowers any medical professional, regardless of their level of sonography training, to accurately assess the position of a mechanical assist device. An artificial intelligence (AI) driven system which performs structural relationship inference may automatically analyze the spatial relationships between key anatomic structures, such as the aortic annulus, septum, and mitral valve, and critical elements of the mechanical assist device, such as its inlet and outlet, as seen in an ultrasound image.

Such a system may provide immediate, objective, and critical information regarding device placement. This may enable continuous assessment of the device's position without the need for a specialized sonographer to be present at all times. This, in turn, enhances patient monitoring, reduces the risks and complications associated with malposition, and facilitates faster, more informed clinical decision-making, including any necessary repositioning of the device.

Applicant has further realized that the images captured via echocardiography may be rapidly and easily shared with the relevant members of the care team. This facilitates remote consultation and decision-making regarding the necessity for repositioning the pump.

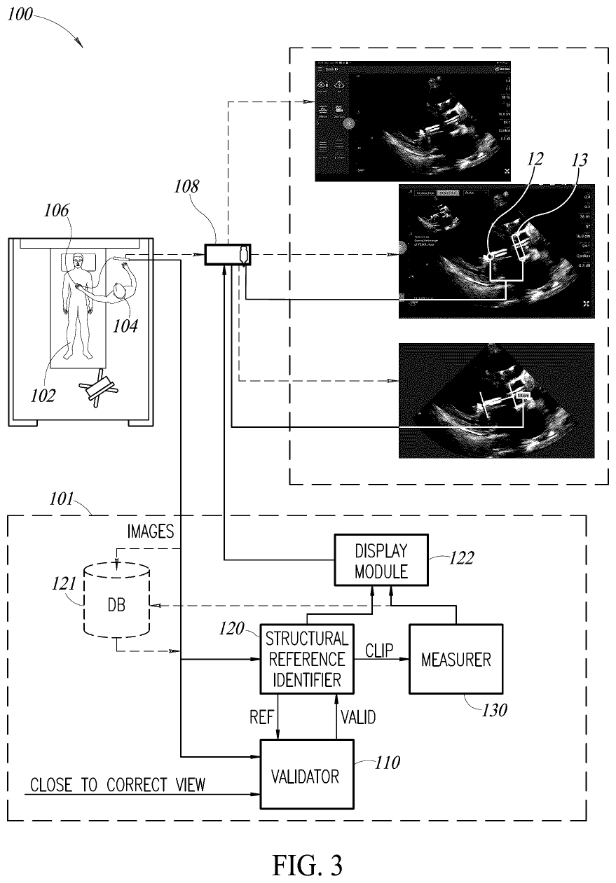

Reference is now made to FIG. 3, which illustrates a system 100 providing ultrasound for mechanical assist devices, in operation on a patient 102 having a mechanical assist device (not shown) in his/her heart.

A medical professional 104, such as a sonographer, may use an ultrasound probe 106 to scan the patient 102, moving the probe around to find the desired “canonical view” used to check the positioning of the mechanical assist device. As mentioned hereinabove, this canonical view is often the PLAX view, though other views, as described hereinbelow, are also used. The probe 106 may acquire a stream of ultrasound images and may display them on a display 108, which may be positioned at the point of care. Probe 106 may be any type of echocardiogram probe, attached to any suitable type of display, such as cart-based probes and their fixed displays, or mobile probes and their mobile display devices, such as tablet computing devices, mobile phones, laptop computers, etc., or any other suitable ultrasound device.

At the same time or at a later time, probe 106 may transmit the images to an echocardiogram unit 101, which may comprise a validator 110, a structural reference identifier 120, a display module 122, a measurer 130, and an optional database 121. Validator 110 may operate together with structural reference identifier 120 to determine if a clip of ultrasound images from probe 106, whether a livestream or a clip stored in database 121, may provide a sufficient basis for quantitative analysis.

Identifier 120, which, in a preferred embodiment, may be a trained neural network, may continuously detect and provide the locations of structural references of the mechanical device as well as the anatomical structural references in each frame of the clip. For mechanical assist devices, the structural references may be the mechanical assist device's inlet 12 and the aortic annulus, here labeled 13.

In one preferred embodiment, identifier 120 may be based on a convolutional neural network described in U.S. Pat. No. 11,478,226, issued Oct. 25, 2022, to Yeda Research and Development Co. Ltd, and New York University and entitled “System and Method for Ultrasound Analysis”, which patent is incorporated herein by reference. In this embodiment, the neural network is trained to identify the relevant structural references, such as inlet 12 and annulus 13, as well as to provide their physical location in 3D space and/or on the image plane.

To ensure the reliability of the system's outputs, validator 110 may operate according to specific, quantifiable criteria. In a preferred embodiment, validator 110 may determine a clip to be “acceptable” for quantitative analysis only if the key structural references, such as device inlet 12 and aortic annulus 13, are simultaneously and clearly visible for a predefined duration. This duration may be at least 200 milliseconds, which corresponds to approximately six frames in a 30 frames-per-second image stream. This level of clarity and temporal continuity may ensure that the subsequent measurement provided by measurer 130, from the location information generated by identifier 120, may be accurate to within a predefined tolerance, which may be 0.5 centimeters. Alternatively, the duration may be defined as 3 seconds and/or having been seen during at least two cardiac cycles. Measurer 130 may optionally store its measurement, as well as the time at which the measurement was taken, in database 121.

For a clip to be valid, validator 110 may require that all key structural references be valid in that clip and may indicate to structural reference identifier 120 the validity or lack thereof for each clip. It will be appreciated that validator 110 may assess all image quality issues, such as acoustic shadowing, dropout, or motion blur, that typically occur when probe 106 may not be viewing the landmarks correctly. These image quality issues affect whether or not one or more of the landmarks are visible.

Typically, once the clip is validated, identifier 120 then provide markings to display module 122 to mark each reference on display 108, such as with an overlay, in its identified location. Alternatively, identifier 120 may provide the markings once the reference structure is identified. For example, in FIG. 3, device inlet 12 may be marked by a circle and aortic annulus 13 may be marked by an elongated bar with endpoints to clearly delineate the annulus.

For offline operation, validator 110 may review the clip and, if no portion of the clip is valid, validator 110 may provide a comment to that effect to display module 122.

The system may also provide for user interaction and oversight. A medical professional 104 may be able to manually mark their own annotations on display 108. In such an instance, any manual identifications may be rendered in a visually distinct manner, for example using a different color, from the automatically generated ones. Upon a manual marking, measurer 130 may automatically recalculate the associated measurement.

During and after validation, structural reference identifier 120 may process the images to continuously detect and delineate the structural references of the implanted device and the relevant anatomical structures. As mentioned hereinabove, for mechanical assist devices, the device structural reference is the device's inlet 12, and the anatomical structural reference is the aortic annulus 13. In a preferred embodiment, aortic annulus 13 may be identified by detecting its two cusps, specifically the non-coronary cusp and the right coronary cusp. Moreover, the elongated bar indicated aortic annulus 13 may connect the identified cusps.

Measurer 130 may receive location data for the identified references from identifier 120. Measurer 130 may then calculate the spatial relationship between these structures, for example by computing an average distance over a predefined period of continuous data. For mechanical assist devices, the predefined period may be 3 seconds, so as to capture 2 heart cycles. This measurement may indicate that the device's inlet is well positioned within the ventricle without any obstructions.

Measurer 130 may provide the measurement to display module 122 which, in turn, may generate a graphical overlay to indicate this calculated distance on display 108, providing medical professional 104 with an objective measurement, whether generated in real-time or from stored clips, to assess the positioning of mechanical assist device within the heart of patient 102.

It may be noted that, when echocardiogram unit 101 may operate on a stored clip, it may operate partially or totally offline. Unit 101 may also operate on a set of clips, taken at different times, to give an overall measurement for the patient.

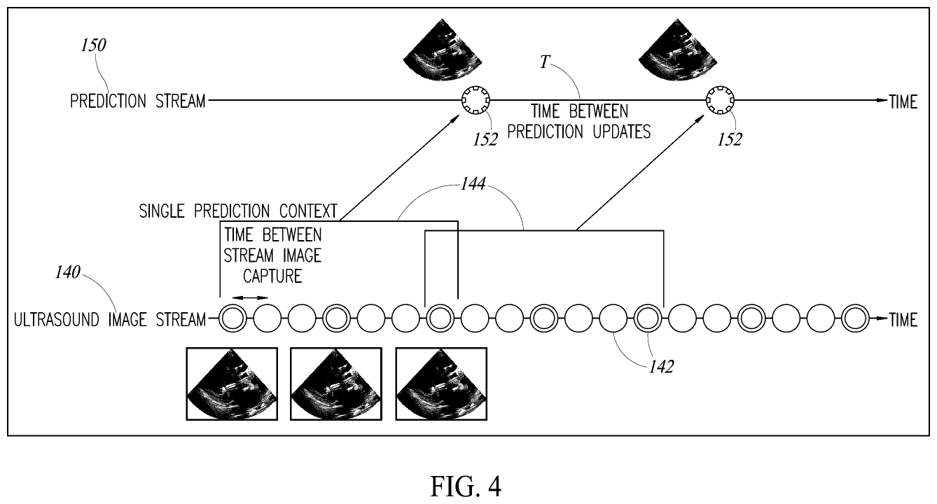

Reference is now made to FIG. 4, which illustrates a data stream of echocardiogram unit 101. The data stream may comprise an ultrasound image stream 140 and a prediction stream 150. Ultrasound image stream 140 may comprise a sequence of individual ultrasound images 142, captured over time.

In a preferred embodiment, structural reference identifier 120 may analyze a group of one or more ultrasound images 142, forming a single prediction context 144, to generate a single prediction 152. Because the system may analyze a context 144 of multiple images, a time T may elapse between each prediction 152 in the prediction stream 150. This use of a prediction context 144 may allow echocardiogram unit 101 to generate more stable and accurate predictions regarding the location of the structural references.

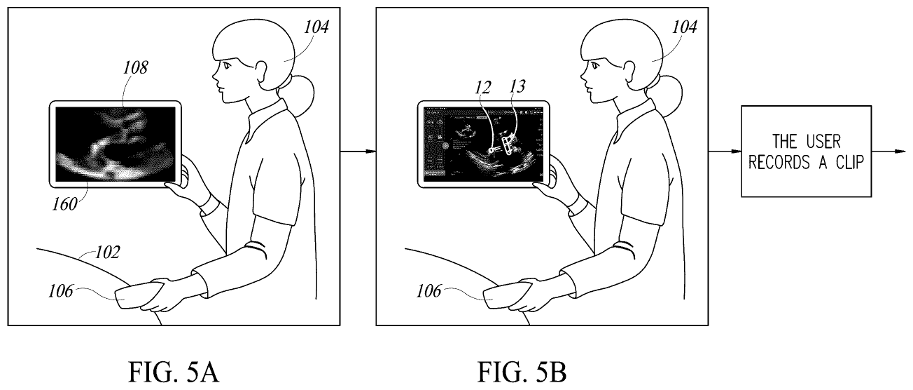

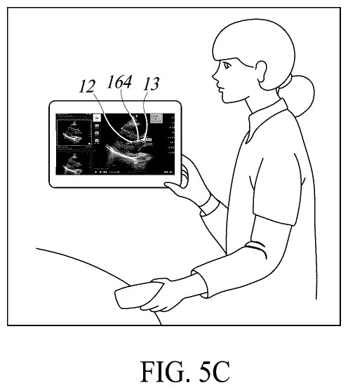

Reference is now made to FIGS. 5A, 5B, and 5C, which illustrate the operation of system 100. In a typical scenario, a medical professional 104, who may not be a trained sonographer, may operate probe 106. This may be during a routine check or in response to an alert from a mechanical assist device, necessitating an echocardiographic assessment to verify the device's position.

As illustrated in FIG. 5A, medical professional 104 may use ultrasound probe 106 to scan patient 102. The professional 104 may move probe 106 to obtain a desired canonical view, such as the PLAX view, a view close to the PLAX view, or any view which shows the device's inlet, which may be displayed in real-time on display 108. FIG. 5A shows an exemplary image 160 that is close to the PLAX view on display 108.

As illustrated in FIG. 5B, as probe 106 approaches the desired view, structural reference identifier 120 may begin to identify the anatomical and device structural references from the live ultrasound image stream. When validator 110 may determine that the structures are valid and clearly visible, as shown on display 108 of FIG. 5B, validator 110 may prompt medical professional 104 to record a clip for analysis or may auto capture a recent sequence of images for later analysis.

As illustrated in FIG. 5C, once a clip is validated, structural reference identifier 120 may re-identify the structures within the clip, and measurer 130 may calculate a distance 164 between identified device inlet 12 and aortic annulus 13. Measurer 130 may then provide calculated distance 164 and the appropriate markings for it (e.g. distance 164 as well as an annotation extending between device inlet 12 and aortic annulus 13) to display module 122, to be presented on display 108, providing an objective measurement to medical professional 104. In certain embodiments, the medical professional 104 may manually mark their own identifications, which may cause measurer 130 to recalculate the measurement for redisplaying on display 108.

Moreover, measurer 130 may provide a score to the measured distance. The score is generated by a binary classifier differentiating between clips where the structures are valid (i.e. visible in the datastream for a predefined length of time) and clips where they are not.

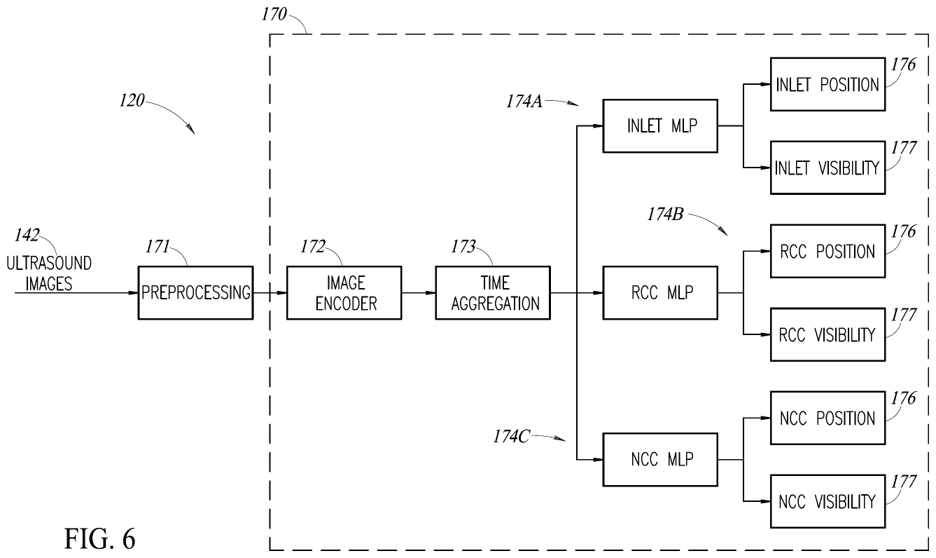

Reference is now made to FIG. 6, which illustrates an exemplary detailed architecture of a combined structural reference identifier 120, which, in this embodiment, may be implemented as a machine learning model, trained for identifying the structural references (anatomical and implanted).

In this embodiment, structural reference identifier 120 may comprise a preprocessing module 171 and a machine learning model 170 comprising an image encoder 172, a time aggregation module 173, and a plurality of prediction heads 174, one per landmark of interest. The input to structural reference identifier 120 may be ultrasound image stream 140. Preprocessing module 171 may receive ultrasound images 142 and may perform operations such as grayscale conversion, image normalization, cropping, and resizing on images 142.

Machine learning model 170 may be implemented with specific architectures to achieve its function. Image encoder 172, which may be a convolutional neural network, may receive the preprocessed images and may process each frame to generate a frame-level feature vector and, optionally, a latent feature of the entire clip. In an exemplary implementation, image encoder 172 may be a convolutional neural network comprising a series of convolutional layers with increasing channel depth, for example from 32 to 512 channels. Prediction heads 174 may be implemented as multi-layer perceptrons (MLPs), which may, for example, comprise two hidden layers each with a dimension of 128.

Time aggregation module 173, which may be a gated recurrent unit, may receive a sequence of the feature vectors from image encoder 172 and may process the sequence to capture temporal dynamics across the entire clip. Time aggregation module 173 may provide the temporal dynamics to the plurality of prediction heads 174, each of which may comprise a position predictor 176 and a visibility predictor 177 for its landmark. Position predictors 176 may implement a regression or a segmentation task, and visibility predictors 177 may implement a binary classification task.

For the example of a mechanical assist device, a first prediction head 174A may generate an inlet position and an inlet visibility status. A second prediction head 174B may generate a right coronary cusp (RCC) position and a right coronary cusp visibility status. A third prediction head 174C may generate a non-coronary cusp (NCC) position and a non-coronary cusp visibility status.

Only clips where the label produced by time aggregation module 173 may indicate that the relevant structural element, annulus or inlet, is visible are provided to each prediction head 174. Prediction heads 174 may then provide their output position information to display module 122 to mark the relevant structural element. Typically, prediction heads 174B and 174C may only output their position information when both cusps are visible according to time aggregation module 173.

Visibility predictors 177 may provide their output to validator 110, which may utilize the visibility predictions to determine which clips, if any, are valid. For most embodiments, validator 110 may require that all predictions be visible for the predefined period of time.

In a preferred embodiment, structural reference identifier 120 may be trained using a dataset of ultrasound clips acquired from a plurality of clinical sites and various ultrasound hardware systems. The training process may begin with the establishment of ground truth data. For this, a panel of expert sonographers may annotate offline ultrasound clips, identifying key structural references, such as the device inlet 12 and the aortic annulus 13 in each relevant frame. A majority agreement or a similar consensus method among the annotators may establish the ground truth labels for both the position and visibility of each landmark. To enhance the robustness and generalization capability of structural reference identifier 120, the training data may undergo a data augmentation process. This process may include applying random transformations to the training images and their corresponding labels. Such transformations may comprise random gamma corrections, within a range of 1.0 to 1.3, to adjust image intensity, random zooms of up to ±10% to handle variations in scale, random translation, of up to ±10% of the image dimensions, to account for shifts in position, and random rotations, of up to 10 degrees, to address in-plane rotational differences.

Structural reference identifier 120 may then be trained by minimizing a composite loss function, which may be a weighted sum of task-specific losses. For instance, a binary cross-entropy loss may be used for the classification task of visibility predictors 177, and a mean absolute error loss may be used for the landmark position regression or segmentation task of position predictors 176. Alternatively, the prediction loss of position predictors 176 may be a DICE loss, typically used for medical segmentation where possibly overlapped shapes are to be matched, with or without a cross-entropy loss. Thus, the position predictors 176 may be trained by comparing a mean prediction of the relevant structural element to a mean label of that element.

This training process may iteratively adjust the model's parameters to accurately predict both the presence and precise location of the structural references based on the annotated ground truth data.

The training process may include performance testing, which utilizes a dataset geographically separated from the training set to prevent bias and to ensure that system 100 may generalize well to new data. The dataset for testing may include echocardiographic clips from a variety of ultrasound devices, to confirm the system's compatibility with different hardware, and from a variety of different patients, in terms of age, gender and BMI (body-mass index).

In other embodiments, identifier 120 may be implemented with any suitable neural network(s), such as convolutional neural networks (CNNs), transformers (ViTs and hybrids), graph neural networks (for structured/relational features), or diffusion models (for generative and representation learning).

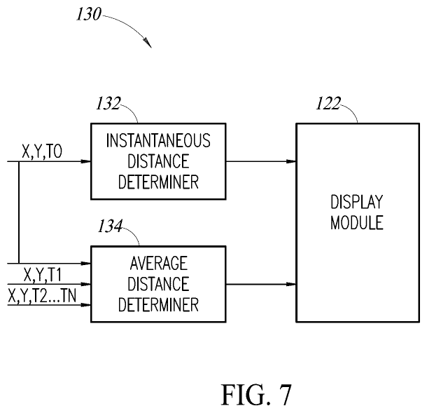

Reference is now made to FIG. 7, which illustrates a detailed architecture of the measurer 130. Measurer 130 may receive location data for the identified structural references from identifier 120. The location data may comprise x and y coordinates for each landmark at a plurality of time points, such as at a first time t0, a second time t1, and up to a final time tN. Measurer 130 may comprise an instantaneous distance determiner 132 and an average distance determiner 134, and may provide their output to display 108. The instantaneous distance determiner 132 may receive location data for a single time point, such as t0, and may compute the spatial distance between the structural references for that specific frame. The average distance determiner 134 may receive location data over a sequence of time points, from t1 to tN, and may compute an average distance over that predefined period. Display 108 may receive the calculated instantaneous and average distances and may display it in the appropriate location on the screen. It will be appreciated that determiners 132 and 134 may allow for the provision of both a real-time distance measurement and a more stable, overall measurement for a clip or a set of clips, respectively.

It will be appreciated that system 100 may provide identification of implanted devices and their structural relationships with anatomical elements.

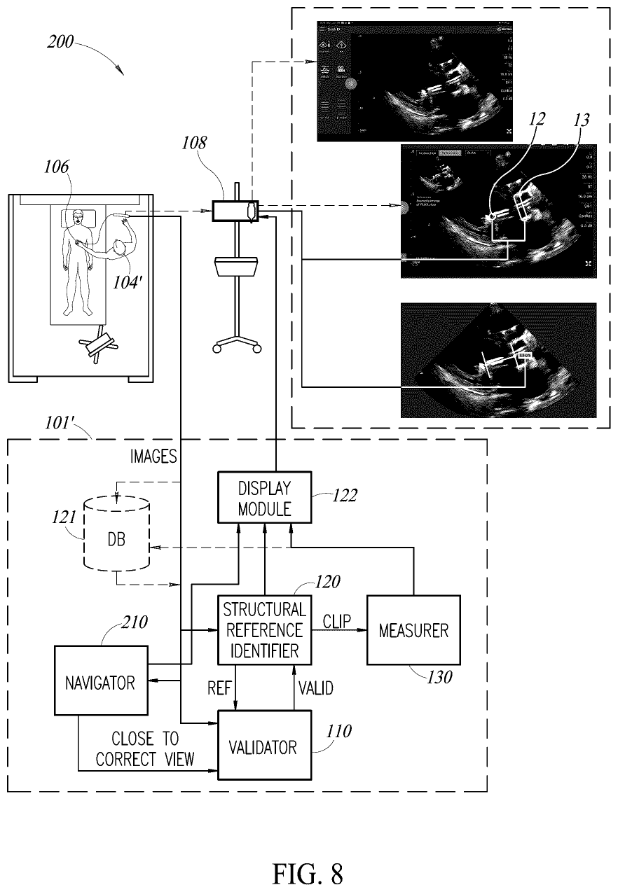

Reference is now made to FIG. 8, which illustrates an alternative system 200 for providing ultrasound for mechanical assist devices. In this embodiment, system 200 may comprise an echocardiogram unit, here labeled 101′, which may comprise an ultrasound navigator 210, in addition to validator 110, structural reference identifier 120, display module 122, and measurer 130.

In this embodiment, display 108 is shown mounted on a medical pole 109, typically used to hang medical fluids near patient 102. Applicant has realized that, within a clinical setting such as an Intensive Care Unit (ICU), a cart-based echocardiogram device utilizes significant floor space, which is at a premium in small, crowded ICU rooms. Moreover, displays 108 which are tablet computing devices or other small displays have no standard place within the ICU. For convenient operation, these small displays should be at the point of care, near patient 102. Applicant has realized that mounting display 108 on medical pole 109 may enhance usability and workflow efficiency.

Navigator 210 may be configured to assist a medical professional who is a non-sonographer, here labeled 104′, in orienting ultrasound probe 106 to acquire a desired canonical view of the heart. Navigator 210 may be the Real Time Guidance software, commercially available from UltraSight Inc . . . of the USA, and described in part in U.S. Pat. No. 11,593,638, issued Feb. 28, 2023, to Yeda Research and Development Co. Ltd, and New York University and entitled “System and Method for Orientating Capture of Ultrasound Images”, which patent is incorporated herein by reference.

Navigator 210 may receive and analyze the live ultrasound image stream 140 from probe 106. Based on this analysis, navigator 210 may generate orientation instructions, which may be presented on display 108, to guide non-sonographer 104′ in adjusting the position and rotation of the probe 106. As non-sonographer 104′ may manipulate the probe according to the instructions, navigator 210 may continuously monitor the incoming images. When navigator 210 may determine that the probe's orientation is sufficiently close to the desired canonical view, it may provide the image stream to validator 110 and to structural reference identifier 120.

According to a preferred embodiment of the present invention, once validator 110 may confirm that a valid clip has been acquired, optionally, navigator 210 may cease providing orientation instructions, thereby enabling non-sonographer 104′ to stabilize the probe for recording or for analysis by validator 110 and structural reference identifier 120. Subsequent processing by structural reference identifier 120 and measurer 130 may then proceed as described hereinabove for system 100.

It will be appreciated that system 200 may empower any medical professional to receive important measurements about mechanical assist device 10. This may ensure 24/7 availability of echocardiograms. Furthermore, with navigator 210, system 200 may empower any medical professional within the ICU environment to accurately place the echocardiogram probe at the relevant view to identify the position of the mechanical assist device 10.

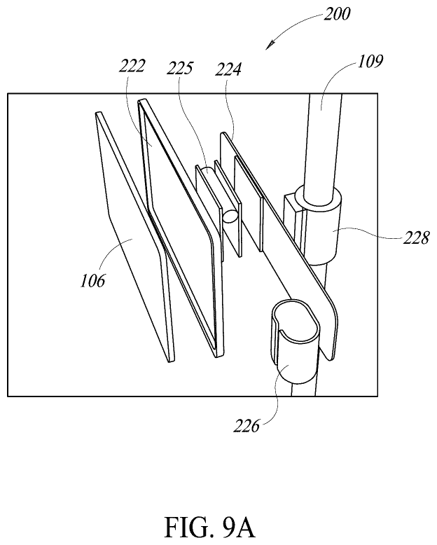

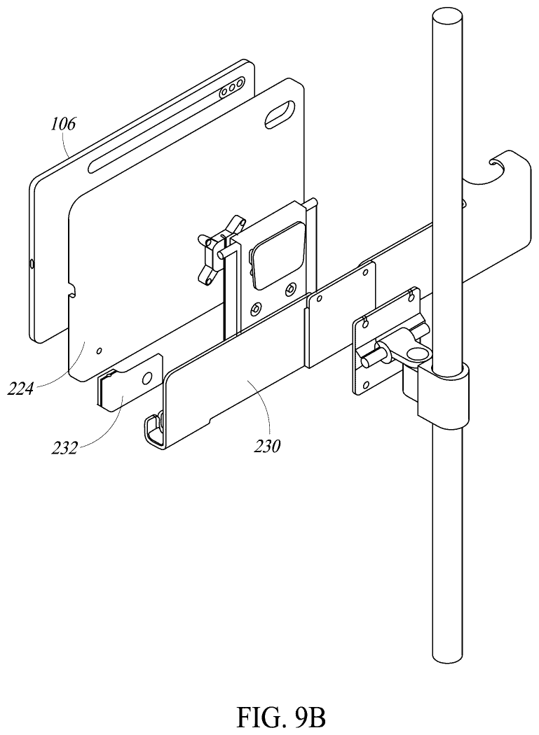

Reference is now made to FIGS. 9A and 9B, which are front and back expanded views, respectively, of a modular mounting assembly 220 for mounting mobile display 108 to medical pole 109. Medical pole 109 may be generally used to hold medical supplies and/or equipment thereon and may or may not have its own display or tablet thereon. Mounting assembly 220 may be connected to medical pole 109 at any suitable height thereon.

Assembly 220 may comprise a tablet case 222, a mounting plate 224 with an integrated transducer holder 226, and a clamp component 228. This modular design may combine custom-fabricated components with standard commercially available parts to provide a versatile and robust mounting solution. In this embodiment, display 108 may be a tablet computer and may be housed within tablet case 222. Tablet case 222 may be configured to quickly connect to and disconnect from mounting plate 224. Mounting plate 224 may comprise a quick connect/disconnect 225 to quickly connect tablet case 222 thereto and to quickly disconnect it therefrom. In addition, mounting plate 224 may be designed to interface with the clamp component 228, which may be a standard clamp, such as a GCX clamp, capable of rotation to accommodate various pole diameters and mounting orientations. Integrated transducer holder 226 may provide a convenient location for storing the ultrasound probe 106 when not in use.

FIG. 9B is a detailed, exploded view of the modular mounting assembly 220. The assembly 220 may further comprise a tablet mounting plate 230 and a transducer security module 232. Tablet case 222 may attach to the tablet mounting plate 230, which may feature an integrated kickstand for standalone use. This sub-assembly may then connect to main mounting plate 224, which may incorporate features for cable management in addition to integrated transducer holder 226. Transducer security module 232 may prevent unauthorized removal or loss of the probe 106. The entire assembly may be secured to standard medical pole 109 by clamp component 228.

Applicant has further realized that, because echocardiogram unit 101 or 101′ may be implemented in the tablet or other mobile computing device of display 108, the tablet may enable other point of care operations. For example, one type of point of care operation may be administrative functions which may include associating the output of echocardiogram unit 101 or 101′ to the relevant patient.

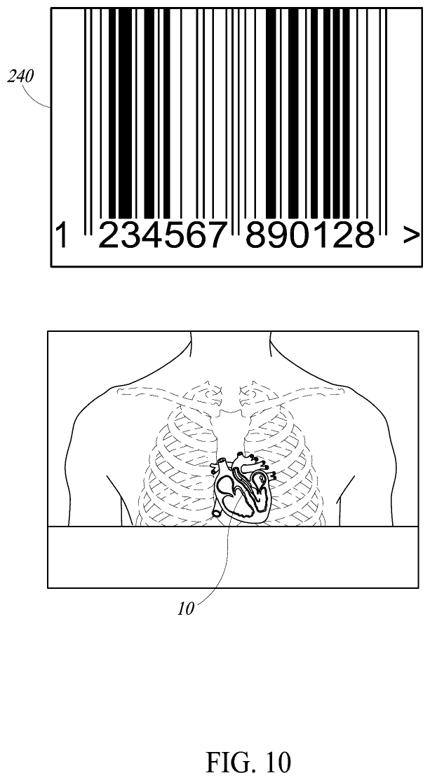

Reference is now made to FIG. 10, which illustrates an embodiment of a device identification function. System 100 or 200 may comprise a barcode scanning functionality to streamline the association of a patient assessment with a specific mechanical assist device. This functionality may utilize an integrated camera of display 108 to capture and decode a barcode 240. Barcode 240 may be affixed to the mechanical assist device or to its packaging and may encode a unique device identifier. This process may allow for the automatic and accurate input of the mechanical assist device's ID into the hospital's information system, reducing the potential for manual entry errors.

In an alternative embodiment, where barcode scanning may not be available, system 100 or 200 may be configured to receive or access a list of currently active mechanical assist devices 10 from a hospital's information system or from an information system of the supplier of assist devices 10. Medical professional 104 may then select the appropriate device from this list on display 108.

Applicant has further realized that, in addition to measuring the distance between the device inlet and the aortic annulus, a more comprehensive assessment of the mechanical assist device's position and rotational orientation relative to surrounding cardiac anatomy may provide critical information. Moreover, displaying a safe zone within that anatomy may ensure proper device function and patient safety.

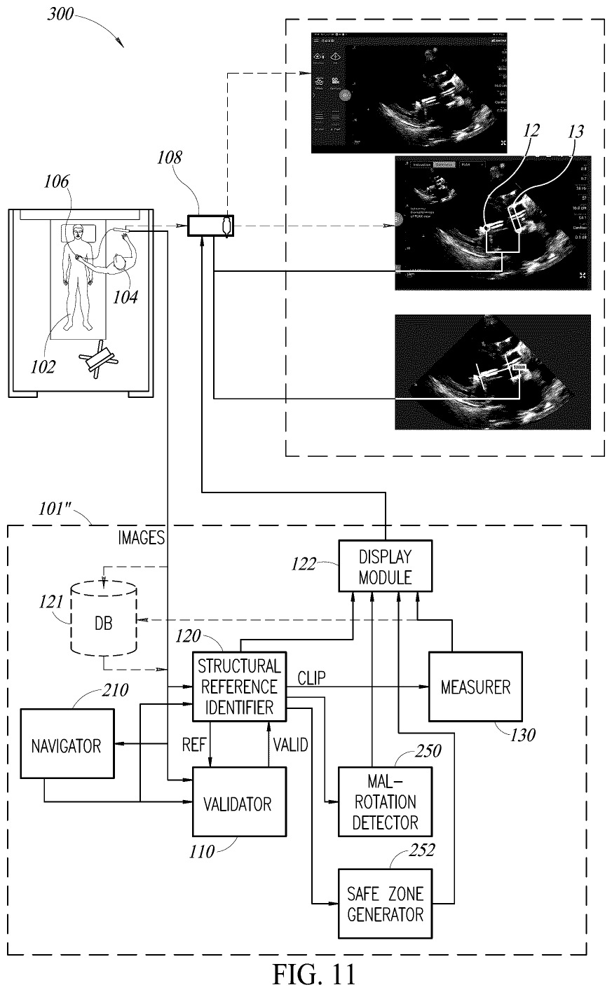

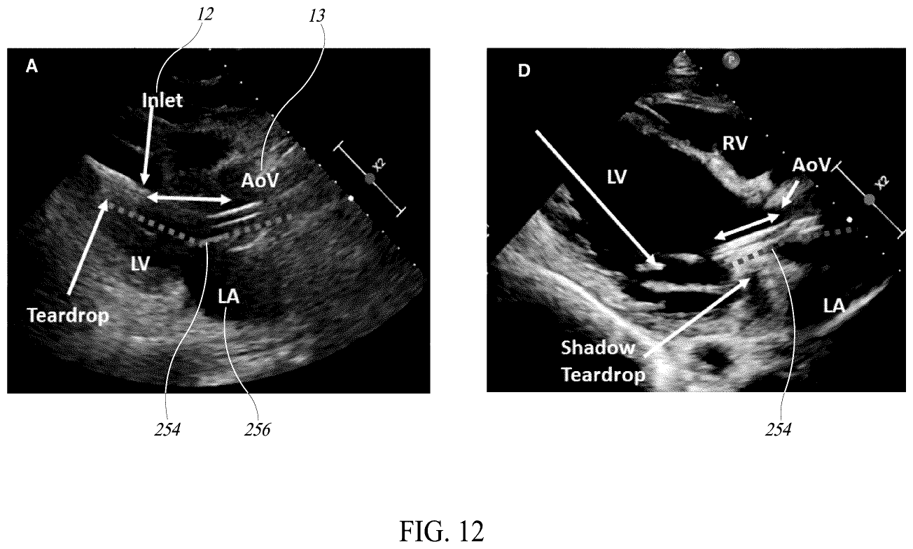

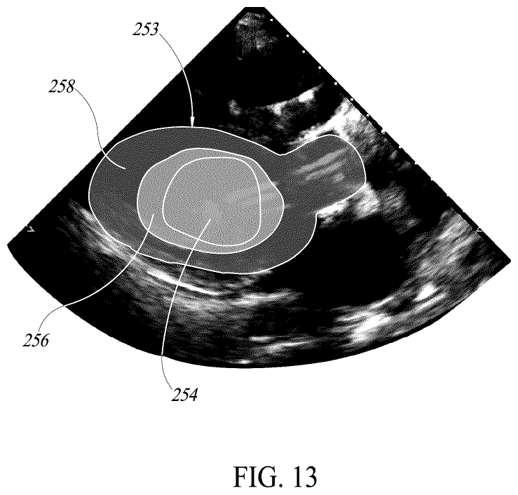

Reference is now made to FIGS. 11, 12, and 13, which together illustrate an embodiment of a system providing advanced positional insights. FIG. 11 illustrates a system 300, which may build upon the architecture of system 200 and may comprise an echocardiogram unit, here labeled 101″, comprising a mal-rotation detector 250 and a safe zone generator 252, along with navigator 210, validator 110, structural reference identifier 120, display module 122, and measurer 130. FIG. 12 illustrates exemplary images of optimal and suboptimal device positioning that system 300 may identify and FIG. 13 illustrates an exemplary output generated by safe zone generator 252 for presentation by display module 260.

The image on the left of FIG. 12 depicts a properly rotated device, with the pump of mechanical assist device, here labelled 254, in the left ventricle (LV) and inlet 12 within aortic valve 13. Note that pump 254 has a concave curve to it.

The image on the right of FIG. 12 depicts a mal-rotated device, where mal-rotation detector 250 may identify that pump 254 has a convex shape, indicating that it is positioned incorrectly, potentially causing an obstruction.

Mal-rotation detector 250 may be implemented as a neural network, such as a binary classifier, trained on a dataset of ultrasound clips that have been expertly labeled as either “correctly rotated”, like the image on the left of FIG. 12, “mal-rotated”, like the image on the right of FIG. 12, or an indication of its depth (optimal depth, too shallow, too deep, full in the left ventricle or in the aortic valve). Mal-rotation detector 250 may identify whether or not mal-rotation is present, and may provide the depth indication.

Safe zone generator 252 may comprise an expanded version of structural reference identifier 120, trained to identify and delineate the boundaries of the left ventricle wall and to identify not only device inlet 12, but also surrounding anatomical structures, such as the left ventricle wall, the mitral valve, the septum, pupillary muscles, and associated leaflets. In addition, safe zone generator 252 may be trained to identify the three zones 254, 256 and 258 within the left ventricle. For this, safe zone generator 252 may be trained with a boundary or contour loss which penalizes a distance between predicted and ground-truth edges, such as of the left ventricle wall and of the surrounding anatomical structures.

Safe zone generator 252 may utilize this information to determine if device inlet 12 currently resides well within the left ventricle and does not touch any parts of the heart, to ensure good fluid flow through inlet 12.

Based on the identification of the cardiac anatomy, safe zone generator 252 may create a map of zones 253 to be overlaid on the ultrasound image, which may be a color-coded map. Map of zones 253 may comprise an inner zone 254, indicating a safe or optimal position for the device inlet; a mid-zone 256, indicating a suboptimal position; and an outer zone 258, indicating an unsafe or prohibited position. The zones may be outlined or filled in, typically with different colors to denote the different zones.

To do so, safe zone generator 252 may identify anatomical elements in the clip and may define the outer edge of outer zone 256 as a closed circle touching the identified anatomical elements.

Safe zone generator 252 may then identify the zone in which pump 254 or inlet 12 is located and may indicate such to display module 122 for displaying on display 108. In an alternative embodiment, safe zone generator 252 may provide a textual indication of how well located (e.g. good/bad/close) inlet 12 is.

System 300 may provide medical professional 104 with feedback to accurately reposition the device into safe zone 254, thereby correcting the mal-position and mal-rotation issues exemplified in FIG. 12 and reducing repositioning errors. It will be appreciated that the feedback may be shown in real time or on stored clips. The latter may provide a more delineated understanding of the position.

Applicant has further recognized that the system's capabilities may be extended beyond positional assessment to include automated, real-time assessment of cardiac function, such as is described in U.S. Pat. No. 11,478,226, discussed hereinabove. Such functional assessment, of left ventricular function, right ventricular function, pericardial effusion and particularly of myocardial contractility, discussed hereinbelow, may be critical for making clinical decisions, such as adjusting the level of mechanical support or determining when a patient's heart has recovered sufficiently to be weaned from the device. Other functional assessments may include diastolic collapse of the right atrium, diastolic collapse of the right ventricle or IVC maximum diameter (cm) and collapsibility index (%).

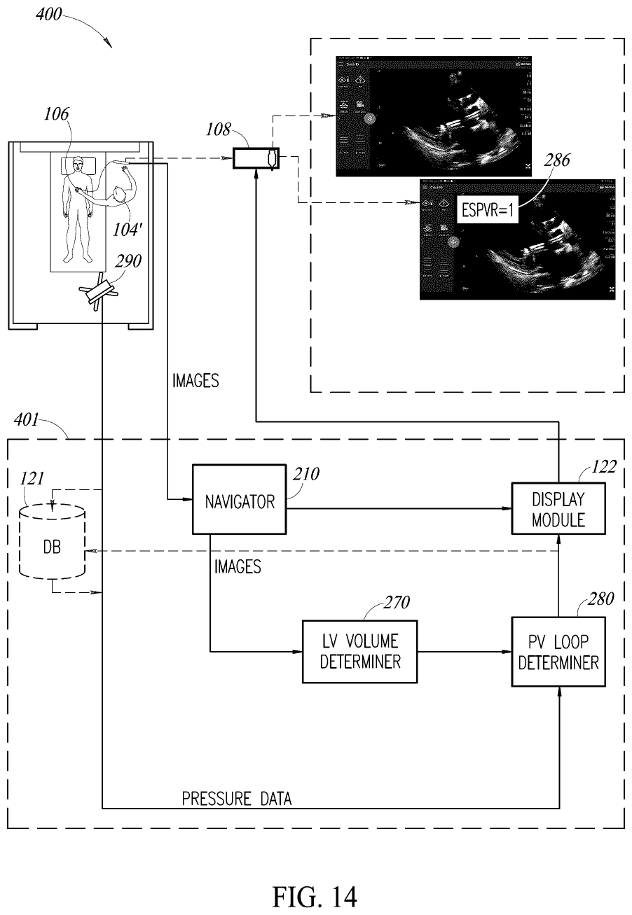

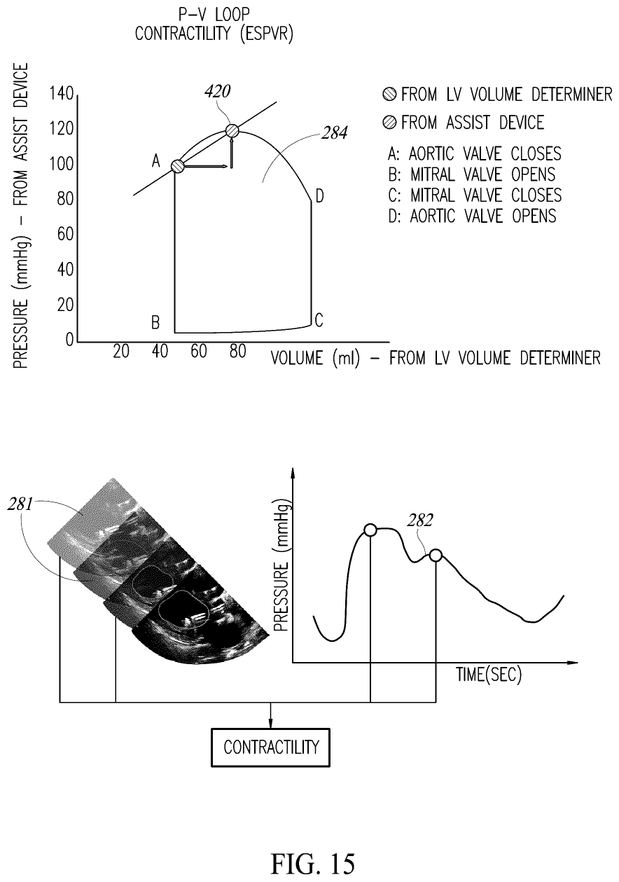

Reference is now made to FIGS. 14 and 15, which together illustrate an embodiment of a system configured for automated contractility assessment. FIG. 14 illustrates a system 400, which may comprise navigator 210 and validator 110. In this embodiment, system 400 may further comprise a left ventricle (LV) volume determiner 270 and a pressure-volume (PV) loop determiner 280. FIG. 15A illustrates the input to PV loop determiner 280 and FIG. 15B illustrates the output and function of PV loop determiner 280.

System 400 may be configured to receive both the ultrasound image stream from probe 106 and a stream of pressure data from the mechanical assist device itself, here labelled 290.

In a preferred embodiment, LV volume determiner 270 may be based on the neural network of U.S. Pat. No. 11,478,226, mentioned hereinabove, which may determine LV volume over time from ultrasound images. LV volume determiner 270 may additionally be trained on such volume signals with indications of when valves open and close. Thus, LV volume determiner 270 may generate a time-stamped volume signal marked with cardiac cycle events.

As shown in FIG. 15A, PV loop determiner 280 may receive the cardiac-cycle-marked volume data from volume reconstructor 272, generated from time-stamped images 281, and may synchronize the volume data to time-varying pressure data 282 from the assist device 290. As FIG. 15A shows, each time-stamped image 281 may be synchronized to a time-stamped pressure level 282.

PV loop determiner 280 may then generate a P-V loop 284, shown in FIG. 15B, by plotting the pressure data against the corresponding volume data for given time points in a cardiac cycle. P-V loop 284 may provide a graphical representation of the heart's mechanical work, indicating key cardiac cycle events such as aortic valve closure (A), mitral valve opening (B), mitral valve-closure (C), and aortic valve opening (D), as determined by cyclical canonical view neural network 274

Furthermore, by analyzing P-V loops generated over multiple cardiac cycles, PV loop determiner 280 may calculate an End-Systolic Pressure-Volume Relationship (ESPVR) as the slope between a point A, the point where the aortic valve closes, and a peak pressure point 420, as measured by assist device 290. The ESPVR may be a robust, load-independent measure of myocardial contractility. Display module 122 may display this contractility value or a trend thereof, in a box 286 on display 108, providing medical professional 104 with a direct, quantitative, and automated assessment of cardiac function at the bedside. This integration of imaging and device data may allow for a more complete physiological assessment, which may aid in critical clinical decision-making.

In further embodiments, the present invention may be configured to operate with a variety of imaging views and device types. While the Parasternal Long-Axis (PLAX) view is described herein as an exemplary canonical view, the invention is not so limited. Structural reference identifier 120 may be trained to identify structural references in other canonical views, such as an Apical 3-Chamber (A3C) view or an Apical 5-Chamber (A5C) view. This may provide flexibility in clinical situations where obtaining a clear PLAX view is difficult. Moreover, the other views may be useful in assessing contractility, LV/RV functions and/or pericardial effusion, as discussed hereinabove.

Furthermore, the underlying principle of structural relationship inference may be applied to other types of implanted medical devices, such as LVAD (left ventricle assist device) and ECMO (extracorporeal membrane oxygenation) devices. For each type of implanted device, structural reference identifier 120 may be trained on images of their inlet devices and their corresponding anatomical landmarks. Measurer 130 may accordingly be configured to calculate other relevant spatial relationships, such as angles or areas, as clinically required. Systems 100, 200, 300 and 400 may also operate on a previously acquired clip of ultrasound images provided from a storage unit, enabling offline or asynchronous analysis in addition to real-time assessment of a live image feed.

Unless specifically stated otherwise, as apparent from the preceding discussions, it is appreciated that, throughout the specification, discussions utilizing terms such as “analyzing,” “generating,” “processing,” “computing,” “calculating,” “determining,” or the like, refer to the action and/or processes of a general purpose computer of any type, such as a client/server system, mobile computing devices, tablets, smart appliances, cloud computing units or similar electronic computing devices that manipulate and/or transform data within the computing system's registers and/or memories into other data within the computing system's memories, registers or other such information storage, transmission or display devices.

The inventive elements discussed hereinabove may be implemented on a suitable apparatus. This apparatus may be specially constructed for the desired purposes, or it may comprise a computing device or system typically having at least one processor and at least one memory, selectively activated or reconfigured by a computer program, code or prompt. The resultant apparatus when instructed by program, code or prompt may turn the general-purpose computer into inventive elements as discussed herein. The program, code or prompt may define the inventive device in operation with the computer platform for which it is desired. Such program, code or prompt may be stored in a computer readable storage medium, such as, but not limited to, any type of disk, including optical disks, magnetic-optical disks, read-only memories (ROMs), volatile and non-volatile memories, random access memories (RAMs), electrically programmable read-only memories (EPROMs), electrically erasable and programmable read only memories (EEPROMs), magnetic or optical cards, Flash memory, disk-on-key or any other type of media suitable for storing programs, code or prompts. The computer readable storage medium may also be implemented in cloud storage.

Some general-purpose computers may comprise at least one communication element to enable communication with a data network and/or a mobile communications network.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the invention as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A unit for assessing a position of an implanted device within a body part, said unit comprising:
a structural reference identifier configured to receive one or more ultrasound images, and to identify and to locate therefrom, at least one anatomical structural reference of said body part and at least one device structural reference of said implanted device.

2. The unit of claim 1, and also comprising a structural reference measurer configured to calculate a spatial relationship between said at least one anatomical structural reference and said at least one device structural reference to provide a positional assessment of said implanted device.

3. The unit of claim 2, wherein said implanted device is a mechanical assist device, said body part is a heart, said at least one anatomical structural reference is an aortic annulus, said device at least one structural reference is an inlet of said mechanical assist device, and said spatial relationship is a distance.

4. The unit of claim 2, wherein said structural reference identifier comprises a machine learning model trained to identify and locate said at least one anatomical and device structural references and to determine a visibility level for said anatomical and device structural references.

5. The unit of claim 4, further comprising a validator to receive said one or more ultrasound images as a sequence of frames and to permit said structural reference measurer to measure said references only when said at least one anatomical structural reference and said at least one device structural reference are determined to both be visible for a predefined duration within said sequence of frames.

6. The unit of claim 2, further comprising a display module configured to generate a graphical overlay on said one or more ultrasound images, said overlay comprising an indication of said spatial relationship.

7. The unit of claim 1, further comprising a display module configured to generate a graphical overlay on said one or more ultrasound images, said overlay comprising a first marker indicating a location of said identified anatomical structural reference and a second marker indicating a location of said identified device structural reference.

8. The unit of claim 1, further comprising an ultrasound navigator to analyze a stream of said one or more ultrasound images and to generate orientation instructions for guiding a user to position an ultrasound probe to acquire a view close to a canonical view of said body part.

9. The unit of claim 3, further comprising a mal-rotation detector to identify one or more surrounding anatomical structures of a left ventricle of said heart within said one or more ultrasound images and to determine whether or not a rotational and/or positional orientation of said implanted device is properly rotated and oriented within said left ventricle.

10. The unit of claim 3, further comprising a safe zone generator trained to identify and delineate boundaries of the left ventricle wall and configured to generate a map of zones overlaid on said one or more ultrasound images, said map of zones indicating safe and unsafe positions for said implanted device.

11. The unit of claim 3 further comprising:
a left ventricle volume determiner configured to generate a time-stamped volume of a left ventricle, marked with cardiac cycle events, from said one or more ultrasound images; and
a pressure-volume loop determiner configured to synchronize said time-stamped volume with received time-stamped pressure data from said mechanical assist device and to generate a pressure-volume loop therefrom, said pressure-volume loop determiner to mark said loop with said cardiac cycle events.

12. The unit of claim 1 implemented on a mobile computing device having a display to display said one or more ultrasound images and mountable at a point of care near a patient having said device implanted therein.

13. The unit of claim 12 wherein said mobile computing device comprises a barcode scanning functionality to associate data in a database with said patient having said device.

14. The unit of claim 1, said structural reference identifier to operate on said one or more ultrasound images in real-time or offline.

15. A modular mounting assembly for a mobile display device, said assembly comprising:
a mounting plate;
a case detachably coupled to said mounting plate and configured to house said mobile display device; and
a clamp component attached to said mounting plate and configured to engage a medical pole.

16. The assembly of claim 15 and also comprising a transducer holder integrated with said mounting plate, said transducer holder configured to receive an ultrasound probe.

17. A method for assessing a position of an implanted device within a body part, The method comprising:
receiving one or more ultrasound images of said body part with said implanted device; and
identifying and locating, from said one or more ultrasound images, at least one anatomical structural reference of said body part and at least one device structural reference of said implanted device.

18. The method according to claim 17, further comprising calculating a spatial relationship between said at least one anatomical structural reference and said at least one device structural reference to provide a positional assessment of said implanted device.

19. The method according to claim 18, wherein said calculating of said spatial relationship comprises calculating a distance between an aortic annulus of a heart and an inlet of a mechanical assist device implanted therein.

20. The method according to claim 18, wherein said identifying and locating are performed using a trained machine learning model, the method further comprising determining, using said trained machine learning model, a visibility level for said anatomical and device structural references.

21. The method according to claim 20, further comprising:
receiving said one or more ultrasound images as a sequence of frames; and
permitting said calculating of said spatial relationship only when said determined visibility level indicates that said at least one anatomical structural reference and said at least one device structural reference are both visible for a predefined duration within said sequence of frames.

22. The method according to claim 18, further comprising generating a graphical overlay on said one or more ultrasound images, said overlay comprising an indication of said calculated spatial relationship.

23. The method according to claim 17, further comprising generating a graphical overlay on said one or more ultrasound images, said overlay comprising a first marker indicating a location of said identified anatomical structural reference and a second marker indicating a location of said identified device structural reference.

24. The method according to claim 17, further comprising:
analyzing a stream of said one or more ultrasound images; and
generating orientation instructions for guiding a user to position an ultrasound probe to acquire a view close to a canonical view of said body part.

25. The method according to claim 19, further comprising:
identifying one or more surrounding anatomical structures of a left ventricle of said heart within said one or more ultrasound images; and
determining whether a rotational or positional orientation of said mechanical assist device is properly rotated and oriented within said left ventricle.

26. The method according to claim 19, further comprising:
identifying and delineating boundaries of a left ventricle wall from said one or more ultrasound images; and
generating a map of zones overlaid on said one or more ultrasound images, said map of zones indicating safe and unsafe positions for said mechanical assist device.

27. The method according to claim 19, further comprising:
generating a time-stamped volume of a left ventricle, marked with cardiac cycle events, from said one or more ultrasound images;
receiving time-stamped pressure data from said mechanical assist device;
synchronizing said time-stamped volume with said received time-stamped pressure data;
generating a pressure-volume loop from said synchronized data; and
marking said loop with said cardiac cycle events.

28. The method according to claim 17, further comprising displaying said one or more ultrasound images on a mobile computing device at a point of care near a patient having said device implanted therein.

29. The method according to claim 28, further comprising scanning a barcode to associate data in a database with said patient.

30. The method according to claim 17, wherein said identifying and locating are performed on said one or more ultrasound images in real-time or offline.
